(19) United States
(12) Patent Application Publication
(10) Pub. No.: US 2025/0236906 A1
(43) Pub. Date: Jul. 24, 2025

(54) NUCLEIC ACID PROBE SETS COMPRISING STEM REGION FOR SAMPLE ANALYSIS

(71) Applicant: 10x Genomics. Inc., Pleasanton, CA (US)

(72) Inventor: Octavian Marian BLOJU, Stockholm (SE)

(21) Appl. No.: 19/028,280

(22) Filed: Jan. 17, 2025

Related U.S. Application Data
(60) Provisional application No. 63/622,920, filed on Jan. 19, 2024.

(51) Int. Cl. C12Q 1/682; C12Q 1/6853

(52) U.S. Cl. CPC C12Q 1/682; C12Q 1/6853

(57) ABSTRACT

The present disclosure relates, in some aspects, to methods and compositions for analyzing a biological sample. In some aspects, the methods comprise use of a probe set comprising a first and second polynucleotide which hybridize to a target nucleic acid in the biological sample and form a circularized probe. In some aspects, the methods and compositions provided herein improve the detection of nucleic acids in a biological sample.

### CROSS-REFERENCE TO RELATED APPLICATIONS

This application claims priority to U.S. Provisional Patent Application No. 63/622,920, filed Jan. 19, 2024, entitled “NUCLEIC ACID PROBE SETS COMPRISING STEM REGION FOR SAMPLE ANALYSIS,” which is herein incorporated by reference in its entirety for all purposes.

### REFERENCE TO AN ELECTRONIC SEQUENCE LISTING

The contents of the electronic sequence listing (202412021800SEQLIST.xml; Size: 2,823 bytes; and Date of Creation: Jan. 14, 2025) is herein incorporated by reference in its entirety.

### FIELD

The present disclosure relates in some aspects to methods and compositions for analyzing a biological sample, such as probes for in situ analysis.

### BACKGROUND

Methods are available for analyzing nucleic acids in a biological sample in situ, such as a cell or a tissue. For instance, advances in single molecule fluorescent hybridization (smFISH) have enabled nanoscale-resolution imaging of RNA in cells and tissues. However, oligonucleotide probe-based assay methods for in situ analysis may suffer from low sensitivity, specificity, and/or detection efficiency and may require careful and laborious optimization. Improved methods for in situ analysis are needed. Provided herein are methods and compositions that address such and other needs.

### SUMMARY

Provided herein are methods for assembling probe sets for use in various applications, such as rolling circle amplification, e.g., for nucleic acid analysis such as in situ sequencing and/or in situ hybridization. In one embodiment, a plurality of polynucleotides of a probe set are assembled, e.g., through hybridization followed by ligation, to form a circularized probe. Typical approaches to generating circularized probes from two or more polynucleotides include ligating the polynucleotides using the target nucleic acid as a template, and using a splint as a template, which can introduce various inefficiencies. As discussed herein, the organization and complementarity of the various regions of the polynucleotides provides structural components for the improved assembly and generation of circularized probes.

In some embodiments, provided herein is a method for analyzing a biological sample, comprising: (a) contacting the biological sample with a probe set comprising a first polynucleotide and a second polynucleotide, wherein: the first polynucleotide comprises a first target binding region and a stem region, wherein the stem region is at one terminus of the first polynucleotide and comprises a second polynucleotide binding sequence and a self-complementary sequence, the second polynucleotide comprises a second target binding region and a second probe region, wherein the second probe region binds to the second polynucleotide binding sequence of the first polynucleotide, the stem region brings a terminus of the first polynucleotide adjacent to a terminus of the second polynucleotide, and the first target binding region binds to a first target region in a target nucleic acid at a location in the biological sample and the second target binding region binds to a second target region in the target nucleic acid; (b) connecting the first polynucleotide and the second polynucleotide to generate a circularized probe at the location in the biological sample, wherein the connecting comprises: (i) ligating the first target binding region and the second target binding region using the target nucleic acid as a template, and (ii) ligating the stem region and the second probe region using the first polynucleotide as a template; (c) using a polymerase to perform rolling circle amplification (RCA) of the circularized probe, thereby generating a rolling circle amplification product (RCP), wherein the polymerase is optionally a Phi29 polymerase; and (d) detecting the RCP at the location in the biological sample, thereby analyzing (e.g., detecting or determining) the target nucleic acid or a sequence thereof in the biological sample. In some embodiments, the first polynucleotide comprises, from 5′ to 3′: the first target binding region, the second polynucleotide binding sequence, and the self-complementary sequence. In some embodiments, the first polynucleotide comprises, from 5′ to 3′: the self-complementary sequence, the second polynucleotide binding sequence, and the first target binding region.

In any of the embodiments herein, the second polynucleotide can comprise, from 5′ to 3′: the second probe region and the second target binding region. In any of the embodiments herein, the second polynucleotide can comprise, from 5′ to 3′: the second target binding region and the second probe region. In any of the embodiments herein, at least one of the ligating in (b)(i) and the ligating in (b)(ii) can require gap filling. In any of the embodiments herein, at least one of the ligating in (b)(i) and the ligating in (b)(ii) may but does not need to require gap filling. In any of the embodiments herein, the contacting in (a) and the connecting in (b) can occur simultaneously. In any of the embodiments herein, the contacting in (a) can occur prior to the connecting in (b).

In any of the embodiments herein, the self-complementary sequence can further comprise a linker. In some embodiments, the linker comprises one or more nucleotide residues. In any of the embodiments herein, the linker can be between about 2 and about 10 nucleotide residues in length. In some embodiments, the linker is 4 nucleotide residues in length. In any of the embodiments herein, the linker can comprise a homopolymeric sequence. In some embodiments, the linker comprises a poly (dT) sequence.

In any of the embodiments herein, the self-complementary sequence can be between about 4 and about 40 nucleotide residues in length. In any of the embodiments herein, the self-complementary sequence can be between about 12 and about 20 nucleotide residues in length.

In any of the embodiments herein, the second polynucleotide binding sequence can be between about 2 and about 18 nucleotide residues in length. In any of the embodiments herein, the second polynucleotide binding sequence can be between about 6 and about 8 nucleotide residues in length. In any of the embodiments herein, the second probe region can be between about 2 and about 18 nucleotide residues in length. In any of the embodiments herein, the second probe region can be between about 6 and about 8 nucleotide residues in length. In any of the embodiments herein, the first target binding region and/or the second target binding region can be between about 5 and about 30 nucleotide residues in length. In any of the embodiments herein, the first target binding region and/or the second target binding region can be about 20 nucleotide residues in length. In any of the embodiments herein, the first target binding region and the second target binding region can be equal in length. In any of the embodiments herein, the first target binding region can be longer or shorter than the second target binding region.

In any of the embodiments herein, the first polynucleotide and/or the second polynucleotide can comprise one or more ribonucleotide residues. In any of the embodiments herein, the first polynucleotide and/or the second polynucleotide can comprise no more than four consecutive ribonucleotide residues. In some embodiments, the first polynucleotide and/or the second polynucleotide comprises a 3′ terminal ribonucleotide residue. In some embodiments, the first polynucleotide and/or the second polynucleotide comprises a 5′ phosphate. In some embodiments, the first polynucleotide and/or the second polynucleotide comprises a 5′ flap configured to be cleaved by a nuclease. In some embodiments, the nuclease is a flap endonuclease 1 (FEN1). In some embodiments, the first polynucleotide and/or the second polynucleotide comprises a barcode region. In some embodiments, the barcode region in the first polynucleotide is nonoverlapping with the stem region, and/or the barcode region in the second polynucleotide is nonoverlapping with the second probe region. In some embodiments, the barcode region in the first polynucleotide is at least partially overlapping with the first probe region, and/or the barcode region in the second polynucleotide is at least partially overlapping with the second probe region.

In some embodiments, the biological sample is contacted with a plurality of probe sets each comprising a first polynucleotide and a second polynucleotide that target a different target nucleic acid.

In some embodiments, the stem regions in the plurality of probe sets are common among first polynucleotides targeting different target nucleic acids, and/or the second probe regions in the plurality of different probe sets are common among second polynucleotides targeting different target nucleic acids. In some embodiments, the stem regions in the plurality of different probe sets are cach associated with a different target nucleic acid, and/or the second probe regions in the plurality of different probe sets are each associated with a different target nucleic acid. In some embodiments, the first polynucleotide comprises, from 5′ to 3′: the first target binding region, an optional first spacer, the second polynucleotide binding sequence and the self-complementary sequence; and the second polynucleotide comprises, from 3′ to 5′: the second target binding region, an optional second spacer, and the second probe region. In some embodiments, the self-complementary sequence comprises a linker. In some embodiments, the ligation in (b)(i) and the ligation in (b)(ii) are performed simultaneously. In some embodiments, the ligation in (b)(i) and the ligation in (b)(ii) are performed sequentially in either order. In some embodiments, the ligation in (b)(i) and the ligation in (b)(ii) are performed with one or more washes between (b)(i) and (b)(ii). In some embodiments, the ligation in (b)(i) and the ligation in (b)(ii) are performed without gap-filling prior to cach ligation. In some embodiments, the ligation in (b)(i) and the ligation in (b)(ii) are performed using different ligases. In some embodiments, the ligation in (b)(i) and the ligation in (b)(ii) are performed using the same ligase. In some embodiments, the ligase(s) is/are selected from the group consisting of a Chlorella virus DNA ligase (PBCV DNA ligase), a T4 RNA ligase, a T4 DNA ligase, and a single-stranded DNA (ssDNA) ligase. In some embodiments, the ligation in (b)(i) is an RNA-templated ligation and the ligation in (b)(ii) is a DNA-templated ligation. In some embodiments, the method further comprises one or more washes between the contacting in (a) and the connecting in (b). In some embodiments of any of the methods, the method further comprises one or more washes between the connecting in (b) and the rolling circle amplification in (c). In some embodiments, the RCP is generated using linear RCA comprising a linear RCA, a branched RCA, a dendritic RCA, or any combination thereof. In some embodiments, the RCP is generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and variants or derivatives thereof.

In some embodiments, the method further comprises contacting the biological sample with a primer prior to the rolling circle amplification in (c). In some embodiments, the target nucleic acid acts as a primer for the rolling circle amplification in (c).

In some embodiments, the method further comprises imaging the biological sample to detect the circularized probe and/or the RCP thereof. In some embodiments, the imaging comprises detecting a signal associated with the circularized probe and/or the RCP thereof at a location in the biological sample.

In some embodiments, the circularized probe and/or the RCP thereof comprise the one or more barcode sequences or complements thereof. In some embodiments, the one or more barcode sequences or complements thereof are detected by: contacting the biological sample with one or more detectably-labeled probes that directly or indirectly hybridize to the one or more barcode sequences or complements thereof, and detecting signals associated with the one or more detectably-labeled probes.

In some embodiments, the biological sample is a tissue sample. In some embodiments, the target nucleic acid is in a cell in the tissue sample. In some embodiments, the method further comprises permeabilizing and/or fixing the tissue sample. In some embodiments, the biological sample is a fixed tissue sample. In some embodiments, the biological sample is a formalin-fixed, paraffin-embedded (FFPE) sample, a frozen tissue sample, or a fresh tissue sample. In some embodiments, the tissue sample is embedded in a matrix. In some embodiments, the tissue sample is embedded in a hydrogel.

In some embodiments, the target nucleic acid comprises a DNA or an RNA. In some embodiments, the target nucleic acid is cDNA or mRNA. In some embodiments, the target nucleic acid is an mRNA.

Further provided herein are methods for analyzing a biological sample, comprising: (a) contacting the biological sample with a first polynucleotide and a second polynucleotide, wherein: the first polynucleotide comprises from 5′ to 3′: a first target binding region and a stem region, wherein the stem region comprises from 5′ to 3′: a second polynucleotide binding sequence, and a self-complementary sequence, the second polynucleotide comprises from 5′ to 3′: a second probe region and a second target binding region, the first target binding region is complementary to and capable of binding to a first target region in a target nucleic acid, the second target binding region is complementary to and capable of binding to a second target region in the target nucleic acid, the second probe region of the second polynucleotide binds to the second polynucleotide binding sequence of the first polynucleotide, and the stem region brings a terminus of the first polynucleotide adjacent to a terminus of the second polynucleotide, (b) ligating the first polynucleotide and the second polynucleotide: (i) at a first ligation site, wherein the first ligation site uses the target nucleic acid as a template and (ii) at a second ligation site, wherein the second ligation site uses the first polynucleotide as a template, wherein ligating the first polynucleotide and the second polynucleotide at the first ligation site and the second ligation site generates a circularized probe at a location in the biological sample; (c) generating a rolling circle amplification product (RCP) by rolling circle amplification of the circularized probe with a polymerase, wherein the polymerase is optionally a Phi29 polymerase; (d) detecting the RCP at the location in the biological sample.

Further provided herein are methods for detecting a target nucleic acid in a biological sample, comprising: (a) contacting the biological sample with a first polynucleotide and a second polynucleotide, wherein: a stem region and a first target binding region, wherein the stem region comprises from 5′ to 3′: a self-complementary sequence and a second polynucleotide binding sequence, the second polynucleotide comprises from 5′ to 3′: a second target binding region and a second probe region, the first target binding region is complementary to and capable of binding to a first target region in the target nucleic acid, the second target binding region is complementary to and capable of binding to a second target region in the target nucleic acid, the second probe region of the second polynucleotide binds to the second polynucleotide binding sequence of the first polynucleotide, and the stem region brings a terminus of the first polynucleotide adjacent to a terminus of the second polynucleotide, (b) ligating the first polynucleotide and the second polynucleotide: (i) at a first ligation site, wherein the first ligation site uses the target nucleic acid as a template and (ii) at a second ligation site, wherein the second ligation site uses the first polynucleotide as a template, wherein ligating the first polynucleotide and the second polynucleotide at the first ligation site and the second ligation site generates a circularized probe at a location in the biological sample; (c) generating a rolling circle amplification product (RCP) by rolling circle amplification of the circularized probe with a polymerase, wherein the polymerase is optionally a Phi29 polymerase; (d) detecting the RCP at the location in the biological sample, thereby detecting the target nucleic acid in the biological sample.

Provided herein is system comprising a probe set comprising a first polynucleotide and a second polynucleotide, wherein the first polynucleotide comprises a first target binding region and a stem region, wherein the stem region is at one terminus of the first polynucleotide and comprises a second polynucleotide binding sequence and a self-complementary sequence, the second polynucleotide comprises a second target binding region and a second probe region, wherein the second probe region is configured to bind to the second polynucleotide binding sequence of the first polynucleotide; a ligase for generating a circularized probe comprising the first polynucleotide and the second polynucleotide; and a polymerase for performing rolling circle amplification (RCA) of the circularized probe. In some embodiments, the kit comprises reagents for detecting a sequence of the first polynucleotide and the second polynucleotide or a complement thereof. In some aspects, the reagents for detecting the sequence of the first polynucleotide and the second polynucleotide or a complement thereof comprises one or more detectably-labeled probes. In some aspects, the reagents for detecting the sequence of the first polynucleotide and the second polynucleotide or a complement thereof comprises reagents for performing sequencing-by-synthesis (SBS), sequencing-by-avidity (SBA) or sequencing-by-binding (SBB). In some aspects, the polymerase is a Phi29 polymerase.

In some aspects, the first polynucleotide or the second polynucleotide comprises one or more barcode sequences. In some instances, the first polynucleotide comprises, from 5′ to 3′: the first target binding region, the second polynucleotide binding sequence, and the self-complementary sequence. In some instances, the first polynucleotide comprises, from 5′ to 3′: the self-complementary sequence, the second polynucleotide binding sequence, and the first target binding region. In some instances, the second polynucleotide comprises, from 5′ to 3′: the second probe region and the second target binding region. In some instances, the second polynucleotide comprises, from 5′ to 3′: the second target binding region and the second probe region. In some instances, the self-complementary sequence further comprises a linker. In some instances, the linker comprises a homopolymeric sequence, optionally wherein the linker comprises a poly (dT) sequence.

### BRIEF DESCRIPTION OF THE DRAWINGS

The drawings illustrate certain features and advantages of this disclosure. These embodiments are not intended to limit the scope of the appended claims in any manner.

FIGS. 1A-1B depict an example of a probe set comprising a first polynucleotide and a second polynucleotide. FIG. 1A depicts the probe set prior to hybridization and ligation, and FIG. 1B depicts the probe set after hybridization and ligations, forming a circularized probe hybridized to the target nucleic acid.

FIG. 2 depicts an example of a stem region of a first polynucleotide (104 and 105) and a second probe region of a second polynucleotide (113). (GCACGCCCGCTGCTTTTGCAGCGGGCGTGC (SEQ ID NO: 1)

### DETAILED DESCRIPTION

All publications, comprising patent documents, scientific articles, and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

When utilizing rolling circle amplification (RCA) to detect nucleic acid targets, two or more polynucleotides may be used in a probe set, where the polynucleotides are connected by ligation to generate a circularized probe. Typical approaches to generating the circularized probes from two or more polynucleotides include ligating the polynucleotides using the target nucleic acid as a template, and using a splint as a template. Splint-templated ligation may have reduced efficiency. In some cases, both ends to be ligated may be occupied by a splint, preventing them from being brought in proximity with each other, thereby preventing ligation. In some cases, the extra hybridization of the splint requires time and steps (e.g., wash step) which contributes to overall reduced efficiency in the workflow. The present application provides improved methods and compositions for generating circularizing probes by splint-free ligation.

Additionally, for detection of nucleic acid targets using RCA, properly resolving densely packed rolling circle amplification products (RCPs) in a biological sample (e.g., a cell) or a surface depends upon resolution of the individual RCPs. RCPs are concatemers comprising multiple copies of the target sequence and the RCA probe sequence, which can be visualized using in situ detection probes or other sequencing methods (e.g., sequencing-by-synthesis (SBS), sequencing-by-avidity (SBA) or sequencing-by-binding (SBB)). RCP detection is enhanced by compaction of the concatemers, as reduction in size results in a higher local concentration of the detection reagents (e.g., detection probes or detectably labeled nucleotides), thereby increasing signal intensity. Compaction also allows for increased resolution of individual RCPs. Typical compaction strategies rely on exogenously added oligonucleotide compaction probes that may be stripped away along with detection probes following rounds of hybridization and target detection. The present application provides methods and compositions for compaction of RCPs without exogenous oligonucleotide compaction probes.

Certain polynucleotides (e.g., a first polynucleotide) of the present disclosure comprise self-complementary sequences designed to form a hairpin structure in a stem region of the polynucleotide. In some embodiments, the stem region comprises a sequence for hybridization to a second polynucleotide, wherein a terminus of the second polynucleotide is brought adjacent to a terminus of the first polynucleotide upon hybridization, which can be ligated together using the first polynucleotide as a template. In some embodiments, the hairpin structure also reduces the overall size of the RCP and reduces the radius of gyration of its diffusion, therefore making the RCP appear smaller and causing decreased optical crowding.

In one aspect, provided herein is a method for analyzing a biological sample, comprising: (a) contacting the biological sample with a probe set comprising a first polynucleotide and a second polynucleotide, wherein: the first polynucleotide comprises a first target binding region and a stem region, wherein the stem region is at one terminus of the first polynucleotide and comprises a second polynucleotide binding sequence and a self-complementary sequence, the second polynucleotide comprises a second target binding region and a second probe region, wherein the second probe region binds to the second polynucleotide binding sequence of the first polynucleotide, the stem region brings a terminus of the first polynucleotide adjacent to a terminus of the second polynucleotide, and the first target binding region binds to a first target region in a target nucleic acid at a location in the biological sample and the second target binding region binds to a second target region in the target nucleic acid; (b) connecting the first polynucleotide and the second polynucleotide to generate a circularized probe at the location in the biological sample, wherein the connecting comprises: (i) ligating the first target binding region and the second target binding region using the target nucleic acid as a template, and (ii) ligating the stem region and the second probe region using the first polynucleotide as a template; (c) using a polymerase to perform rolling circle amplification (RCA) of the circularized probe, thereby generating a rolling circle amplification product (RCP), wherein the polymerase is optionally a Phi29 polymerase; and (d) detecting the RCP at the location in the biological sample, thereby analyzing (e.g., detecting or determining) the target nucleic acid or a sequence thereof in the biological sample.

The composition and methods disclosed herein allow for splint-free ligation, thereby increasing the efficiency of the generation of circularized probes. In some embodiments, the composition and methods disclosed herein allow for compaction of RCPs. In some aspects, provided herein are methods and compositions for splint-free ligation to generate circularized probes and compaction of RCPs using a first polynucleotide and a second polynucleotide, wherein the first polynucleotide comprises a self-complementary sequence that is capable of forming a hairpin structure and a second polynucleotide binding sequence that is capable of binding to the second polynucleotide.

In some embodiments, the first polynucleotide and the second polynucleotide both hybridize to a target nucleic acid and the first polynucleotide and the second polynucleotide also hybridize to each other. In some embodiments, upon hybridization, the termini of the first and second polynucleotides are positioned adjacent to each other. In some embodiments, the termini of the first and second polynucleotides are then ligated, using the first polynucleotide as a template. In some embodiments, the other termini of the first and second polynucleotides are also ligated, using the target nucleic acid as a template. In some embodiments, the hybridization and the ligation occur simultaneously.

Overall, the increased ligation efficiency, compaction of the RCPs, and/or elimination of the need for a ligation splint facilitate the improved detection of target nucleic acids.

### II. Circularized Probes and Products Thereof Comprising Hairpin Structures

Provided herein is a probe set comprising a first polynucleotide and a second polynucleotide, and methods for generating a circularized probe from the first polynucleotide and a second polynucleotide.

### A. Probe Sets for Generating Circularized Probes Comprising Hairpin Structures

In one aspect, the present disclosure provides a composition for sample analysis, including analysis of nucleic acid (e.g., RNA or DNA, such as mRNA or cDNA) and/or non-nucleic acid analytes (e.g., lipids or proteins) in situ in a biological sample. In some embodiments, the composition comprises a first polynucleotide and a second polynucleotide, wherein the first polynucleotide comprises a first target binding region and a stem region, wherein the stem region is at one terminus of the first polynucleotide and comprises a second polynucleotide binding sequence and a self-complementary sequence, the second polynucleotide comprises a second target binding region and a second probe region, wherein the second probe region binds to the second polynucleotide binding sequence of the first polynucleotide, the stem region brings a terminus of the first polynucleotide adjacent to a terminus of the second polynucleotide, and the first target binding region binds to a first target region in a target nucleic acid at a location in the biological sample and the second target binding region binds to a second target region in the target nucleic acid. In some embodiments, the composition comprises plurality of probe sets, wherein each probe set comprises a first polynucleotide and a second polynucleotide, wherein each of the first polynucleotides of each probe set comprises a first target binding region and a stem region.

In some embodiments, the first polynucleotide comprises, from 5′ to 3′: the first target binding region, the second polynucleotide binding sequence, and the self-complementary sequence. In some embodiments, the first polynucleotide comprises, from 5′ to 3′: the self-complementary sequence, the second polynucleotide binding sequence, and the first target binding region.

In some embodiments, the second polynucleotide comprises, from 5′ to 3′: the second probe region and the second target binding region. In some embodiments, the second polynucleotide comprises, from 5′ to 3′: the second target binding region and the second probe region.

In some embodiments, the first polynucleotide comprises, from 5′ to 3′: the first target binding region, the second polynucleotide binding sequence, and the self-complementary sequence, and the second polynucleotide comprises, from 5′ to 3′: the second probe region and the second target binding region.

In some embodiments, the first polynucleotide comprises, from 5′ to 3′: the self-complementary sequence, the second polynucleotide binding sequence, and the first target binding region, and the second polynucleotide comprises, from 5′ to 3′: the second target binding region and the second probe region.

In some embodiments, the first polynucleotide comprises, from 5′ to 3′: the first target binding region the second polynucleotide binding sequence. In some embodiments, the first polynucleotide comprises, from 5′ to 3′: the second polynucleotide binding sequence and the first target binding region.

In some embodiments, the second polynucleotide comprises a stem region. In some embodiments, the second polynucleotide comprises, from 5′ to 3′: a self-complementary sequence, the second probe region and the second target binding region. In some embodiments, the second polynucleotide comprises, from 5′ to 3′: the second target binding region, the second probe region and a self-complementary sequence.

In some embodiments, the first polynucleotide comprises, from 5′ to 3′: the first target binding region and the second polynucleotide binding sequence, and the second polynucleotide comprises, from 5′ to 3′: the self-complementary sequence, the second probe region and the second target binding region.

In some embodiments, the first polynucleotide comprises, from 5′ to 3′: the second polynucleotide binding sequence and the first target binding region, and the second polynucleotide comprises, from 5′ to 3′: the second target binding region, the second probe region and the self-complementary sequence. In some embodiments, upon hybridization of the second probe region and the second polynucleotide binding region, a terminus of the first polynucleotide and a terminus of the second polynucleotide are immediately adjacent to each other.

In some embodiments, the stem region is at one terminus of the first polynucleotide and comprises a self-complementary sequence, which is capable of hybridizing to itself and thus forming a hairpin structure. In some embodiments, the self-complementary sequence is at one terminus of the second polynucleotide and hybridizes to itself and thus forming a hairpin structure. In some embodiments, the self-complementary sequence comprises a palindromic sequence. The self-complementary sequence does not need to comprise a palindromic sequence, as long as it is able to self-hybridize.

In some embodiments, the self-complementary sequence comprises a first portion and a second portion, wherein the first portion is complementary to the second portion, and the first and second portions are separated by a linker. In some embodiments, the linker comprises one or more nucleotide residues. In some embodiments, the linker is between about 2 and about 10 nucleotide residues in length, such as any of 2, 3, 4, 5, 6, 7, 8, 9, or 10 nucleotides residues in length. In some embodiments, the linker is 4 nucleotide residues in length. In some embodiments, the linker comprises a homopolymeric sequence. In some embodiments, the linker is a poly (dT) sequence, optionally a poly (dT) sequence 4 nucleotide residues in length (i.e., TTTT). In some embodiments, the linker is not able to hybridize to the rest of the self-complementary sequence. Therefore, upon hybridization, the self-complementary sequence forms a “stem loop” structure, wherein the first portion and the second portion form the stem, and the linker forms the loop.

In some embodiments, the self-complementary sequence is between about 4 and about 40 nucleotide residues in length. In some embodiments, the self-complementary sequence between about 12 and about 20 nucleotide residues in length. In some embodiments, the linker is 4 nucleotide residues in length. In some embodiments, the first portion and the second portion is cach about 4 nucleotide residues, about 5 nucleotide residues, about 6 nucleotide residues, about 7 nucleotide residues, or about 8 nucleotide residues in length.

In some embodiments, the second polynucleotide binding sequence is between about 2 and about 18 nucleotide residues in length. In some embodiments, the second polynucleotide binding sequence is between about 6 and about 8 nucleotide residues in length. In some embodiments, the second probe region is between about 2 and about 18 nucleotide residues in length. In some embodiments, the second probe region is between about 6 and about 8 nucleotide residues in length. In some embodiments, the second polynucleotide binding sequence and the second probe region are of equal length. The second probe region is complementary to the second polynucleotide binding sequence of the first polynucleotide and is capable of binding to the second polynucleotide binding region of the first polynucleotide. In some embodiments, the second probe region hybridizes to the second polynucleotide binding sequence of the first polynucleotide.

In some embodiments, a hybridization product comprising the pairing of substantially complementary or complementary nucleic acid sequences within two different molecules is analyzed. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Upon hybridization of the second probe region and the second polynucleotide binding region, a terminus of the first polynucleotide is brought adjacent to a terminus of the second polynucleotide. For example, in some embodiments, the first polynucleotide comprises, from 5′ to 3′: the first target binding region, the second polynucleotide binding sequence, and the self-complementary sequence, and the second polynucleotide comprises, from 5′ to 3′: the second probe region and the second target binding region. Upon hybridization of the second probe region and the second polynucleotide binding region, the 3′ end of the first polynucleotide is brought adjacent to the 5′ end of the second polynucleotide. In other embodiments, the first polynucleotide comprises, from 5′ to 3′: the self-complementary sequence, the second polynucleotide binding sequence, and the first target binding region, and the second polynucleotide comprises, from 5′ to 3′: the second target binding region and the second probe region. Upon hybridization of the second probe region and the second polynucleotide binding region, the 5′ end of the first polynucleotide is brought adjacent to the 3′ end of the second polynucleotide.

In some embodiments, upon hybridization of the second probe region and the second polynucleotide binding region, a terminus of the first polynucleotide and a terminus of the second polynucleotide are immediately adjacent to each other before ligation. In some embodiments, upon hybridization of the second probe region and the second polynucleotide binding region, a terminus of the first polynucleotide and a terminus of the second polynucleotide are not immediately adjacent to each other e.g., separated by one or more intervening nucleotides or “gaps”.

The first target binding region is capable of binding to a first target region in a target nucleic acid at a location in the biological sample and the second target binding region is capable of binding to a second target region in the target nucleic acid. The first target binding region and the second target binding region may be equal or different in length. In some embodiments, the first target binding region and the second target binding region are equal in length. In some embodiments, the first target binding region is longer than the second target binding region. In some embodiments, the first target binding region is shorter than the second target binding region. In some embodiments, the first target binding region and/or the second target binding region is between about 5 and about 30 nucleotide residues in length, such as about 5 nucleotide residues, about 10 nucleotide residues, about 15 nucleotide residues, about 20 nucleotide residues, about 25 nucleotide residues, or about 30 nucleotide residues in length. In some embodiments, the first target binding region and/or the second target binding region is about 20 nucleotide residues in length.

In some embodiments, the first polynucleotide and/or the second polynucleotide comprises one or more ribonucleotide residues. In some embodiments, the ribonucleotide residues are consecutive. In some embodiments, the first polynucleotide and/or the second polynucleotide comprises no more than four consecutive ribonucleotide residues. In some embodiments, the first polynucleotide and/or the second polynucleotide comprises one ribonucleotide residue. In some embodiments, the first polynucleotide and/or the second polynucleotide comprises two consecutive ribonucleotide residues. In some embodiments, the first polynucleotide and/or the second polynucleotide comprises three consecutive ribonucleotide residues. In some embodiments, the first polynucleotide and/or the second polynucleotide comprises four consecutive ribonucleotide residues.

In some embodiments, the one or more ribonucleotide residues are at and/or near a ligatable 3′ end of the first polynucleotide and/or the second polynucleotide. In some embodiments, the first polynucleotide and/or the second polynucleotide comprises a 3′ terminal ribonucleotide residue. In some embodiments, the first polynucleotide and/or the second polynucleotide comprises a 5′ phosphate. For example, in some embodiments, the first polynucleotide comprises a 5′ phosphate and the second polynucleotide comprises a 3′ terminal ribonucleotide residue. In some embodiments, the second polynucleotide comprises a 5′ phosphate and the first polynucleotide comprises a 3′ terminal ribonucleotide residue.

In some embodiments, upon hybridization, the ends of the first target binding region and the second target binding region are immediately adjacent to each other before ligation. In some embodiments, upon hybridization, the ends of the first target binding region and the second target binding region are not immediately adjacent to each other e.g., separated by one or more intervening nucleotides or “gaps”.

In some embodiments, the first polynucleotide and/or the second polynucleotide comprises a 5′ flap configured to be cleaved by a nuclease, optionally wherein the nuclease is a flap endonuclease 1 (FEN1).

In some embodiments, the first polynucleotide and/or the second polynucleotide comprises a barcode region. In some embodiments, the barcode region in the first polynucleotide is nonoverlapping with the stem region, and/or the barcode region in the second polynucleotide is nonoverlapping with the second probe region. In some embodiments, the barcode region in the first polynucleotide is at least partially overlapping with the stem region. In some embodiments, the barcode region in the second polynucleotide is at least partially overlapping with the second probe region.

In some embodiments, the biological sample is contacted with a plurality of different probe sets each comprising a first polynucleotide and a second polynucleotide that target a different target nucleic acid. In some embodiments, the stem regions in the plurality of probe sets are common among first polynucleotides targeting different target nucleic acids, and/or the second probe regions in the plurality of different probe sets are common among second polynucleotides targeting different target nucleic acids. In some embodiments, the stem regions in the plurality of different probe sets are each associated with a different target nucleic acid, and/or the second probe regions in the plurality of different probe sets are each associated with a different target nucleic acid.

In some embodiments, the first polynucleotide and/or the second polynucleotide further comprises a spacer. Therefore in some embodiments, the first polynucleotide comprises, from 5′ to 3′: the first target binding region, an optional first spacer, the second polynucleotide binding sequence and the self-complementary sequence, optionally wherein the self-complementary sequence comprises a linker and the second polynucleotide comprises, from 3′ to 5′: the second target binding region, an optional second spacer, and the second probe region.

In some embodiments, the method further comprises connecting the ends of the first polynucleotide and the second polynucleotide to generate a circularized probe. In some embodiments, the connecting occurs simultaneously with the hybridization (e.g., the hybridization between the first polynucleotide and the target nucleic acid, between the second polynucleotide and the target nucleic acid, and between the first polynucleotide and the second polynucleotide). In some embodiments, the connecting occurs after the hybridization.

In some embodiments, the connecting comprises (i) ligating the first target binding region and the second target binding region using the target nucleic acid as a template, and (ii) ligating the stem region and the second probe region using the first polynucleotide as a template. In some embodiments, the connecting comprises (i) ligating the first target binding region and the second target binding region using the target nucleic acid as a template, and (ii) ligating the stem region and the second polynucleotide binding sequence using the second polynucleotide as a template. In some embodiments, connecting the first polynucleotide and the second polynucleotide forms a circularized probe hybridized to the target nucleic acid.

In some embodiments, the ligation comprises the joining of a 5′ phosphate and a 3′ hydroxyl of the first polynucleotide and the second polynucleotide. In some embodiments, the ligation is performed using any other suitable technique.

In some embodiments, the ligation involves template dependent ligation. For example, the ligating of the first target binding region and the second target binding region uses the target nucleic acid as a template and the ligating of the stem region and the second probe region uses the first polynucleotide as a template. In some embodiments, the ligation involves template independent ligation. In some embodiments, the ligating of the first target binding region and the second target binding region comprises an RNA-templated ligation and the ligating of the stem region and the second probe region comprises a DNA-templated ligation.

In some embodiments, the ligation involves chemical ligation. In some embodiments, the ligation involves enzymatic ligation.

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76(14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate the first polynucleotide and the second polynucleotide together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation of the first target binding region and the second target binding region using the target nucleic acid as a template and the ligation of the stem region and the second probe region using the first polynucleotide as a template e.g., in (b)(i) and (b)(ii) respectively, may involve the use of the same ligase or different ligases. In some embodiments, the ligase(s) is/are selected from the group consisting of a Chlorella virus DNA ligase (PBCV DNA ligase), a T4 RNA ligase, a T4 DNA ligase, and a single-stranded DNA (ssDNA) ligase. For example, the ligation in (b)(i) may involve the use of a splintR ligase and the ligation in (b)(ii) may involve the use of a T4 DNA ligase. In some embodiments, the ligation of the first target binding region and the second target binding region using the target nucleic acid as a template and the ligation of the stem region and the second probe region using the first polynucleotide as a template e.g., in (b)(i) and (b)(ii) respectively, may involve the use of the same ligase or different ligases. In some embodiments, the ligation of the first target binding region and the second target binding region and the ligation of the stem region and the second probe region uses two different ligases (e.g., a first ligase for RNA-templated ligation and a second ligase for DNA-templated ligation). In some embodiments, the ligation of the first target binding region and the second target binding region and the ligation of the stem region and the second probe region is performed at two different temperatures. In some embodiments, the ligation of the first target binding region and the second target binding region and the ligation of the stem region and the second probe region are both performed at room temperature. In some embodiments, the ligation of the stem region and the second probe region is performed at room temperature. In some embodiments, the ligation of the first target binding region and the second target binding region is performed at room temperature.

In some embodiments, the connecting of the ends of the first polynucleotide and the second polynucleotide comprising the ligating of the first target binding region and the second target binding region using the target nucleic acid as a template and/or the ligating of the stem region and the second probe region using the first polynucleotide as a template e.g., in (b)(i) and/or (b)(ii), respectively, comprises direct ligation. In some embodiments, the connecting of the ends of the first polynucleotide and the second polynucleotide in comprises indirect ligation, such as via the intermediacy of one or more gap or gap-filling oligonucleotides or by the extension of the 3′ end of the oligonucleotide probe to fill the gap. In some embodiments, the connecting of the ends of the first polynucleotide and the second polynucleotide comprises ligating the ends of the first polynucleotide and the second polynucleotide to at least one gap (oligo) nucleotide, such that the gap (oligo) nucleotide becomes incorporated into the resulting circular product hybridized to the target nucleic acid. In some embodiments, the ligating of the first target binding region and the second target binding region using the target nucleic acid as a template and/or the ligating of the stem region and the second probe region using the first polynucleotide as a template is preceded by gap filling. In other embodiments, the ligating of the first target binding region and the second target binding region using the target nucleic acid as a template and/or the ligating of the stem region and the second probe region using the first polynucleotide as a template does not require gap filling. In some embodiments, the first target region is 5′ to the second target region in the target nucleic acid. In some embodiments, the first target region is 3′ to the second target region in the target nucleic acid.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire content of which is incorporated herein by reference in its entirety). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

In some embodiments, the ligating of the first target binding region and the second target binding region using the target nucleic acid as a template and the ligating of the stem region and the second probe region using the first polynucleotide as a template (e.g., the ligation in (b)(i) and (b)(ii)) occur simultaneously. In some embodiments, the ligating of the first target binding region and the second target binding region occurs before the ligating of the stem region and the second probe region. In some embodiments, the ligating of the first target binding region and the second target binding region occurs after the ligating of the stem region and the second probe region. In some embodiments, there are one or more washes between the ligating of the first target binding region and the second target binding region and the ligating of the stem region and the second probe region.

In some embodiments, the connecting of the ends of the first polynucleotide and the second polynucleotide e.g., the ligating of the first target binding region and the second target binding region using the target nucleic acid as a template and/or the ligating of the stem region and the second probe region using the first polynucleotide as a template is preceded by one or more washes after the contacting of the biological sample with the probe set. In some embodiments, the connecting is followed by one or more washes before further processing the sample. In some embodiments, a first ligation is performed, the sample is washed, then a second ligation is performed using a different ligase.

### B. Generation of Amplification Products of Circularized Probes Comprising Hairpin Structures

The method disclosed herein comprises generating amplification products of circularized probes comprising hairpin structures. In some embodiments, such amplification products, such as a rolling circle amplification product (RCP) are compacted in size. For example, the RCPs generated using circularized probes comprising hairpin structures as templates are more compact in size compared to RCPs generated using circularized probes without any hairpin structures as template. In some embodiments, a connected (e.g., ligated) first and second polynucleotide of a probe set as described in Section II.A. is a circularized probe and the circularized probe is used a template to perform RCA.

In some embodiments, a primer extension product of an analyte, a labeling agent, a probe set bound to the analyte (e.g., a circularizable probe bound to genomic DNA, mRNA, or cDNA), or a probe set bound to the labeling agent (e.g., a circularizable probe bound to one or more reporter oligonucleotides from the same or different labeling agents) is analyzed.

A primer is generally a single-stranded nucleic acid sequence having a 3′ end that, in some embodiments, is used as a substrate for a nucleic acid polymerase in a nucleic acid extension reaction. The primer, or “priming strand” can be an exogenous primer that is added to the biological sample, or an endogenous nucleic acid. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. In some embodiments, enzymatic extension is performed by an enzyme including, but not limited to, a polymerase and/or a reverse transcriptase.

In some embodiments, a product of an endogenous analyte and/or a labeling agent is an amplification product of one or more polynucleotides, for instance, a first polynucleotide and a second polynucleotide. In some embodiments, the amplifying is achieved by performing rolling circle amplification (RCA). In some embodiments, an endogenous nucleic acid or fragment thereof hybridized to the circularized probe (e.g., one that is formed by the first polynucleotide and the second polynucleotide) is used to prime amplification. In some embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, amplification of a circularizable probe set, e.g., as described in Section II.A, is primed by the target nucleic acid (e.g., target RNA). The target nucleic acid (e.g., target RNA) can optionally be immobilized in the biological sample. In some embodiments, the target nucleic acid (e.g., target RNA) is cleaved by an enzyme (e.g., RNase H). In some embodiments, the target nucleic acid (e.g., target RNA) is cleaved at a position downstream of the target sequences bound to the circularizable probe set (e.g., the connected first polynucleotide and the second polynucleotide). In some aspects, the methods disclosed herein allow targeting of RNase H activity to a particular region in a target RNA that is adjacent to or overlapping with a target sequence for a probe set. For example, a nucleic acid oligonucleotide is designed to hybridize to a complementary oligonucleotide hybridization region in the target RNA. In some embodiments, a nucleic acid oligonucleotide is used to provide a DNA-RNA duplex for RNase H cleavage of the target RNA in the DNA-RNA duplex. In some embodiments, the oligonucleotide binds to the target RNA at a position that overlaps with the target sequence(s) of the probe set by about 1 to about 20 nucleotides or by about 8 to about 10 nucleotides. The cleaved target RNA itself can then be used to prime RCA of the circular probe generated from the circularizable probe set (e.g., the first polynucleotide and the second polynucleotide) (e.g., target-primed RCA). In some cases, a plurality of nucleic acid oligonucleotides can be used to perform target-primed RCA for a plurality of different target RNAs.

In any of the embodiments herein, the biological sample is contacted with the RNase H (and optionally with the nucleic acid oligonucleotide) before or during formation of the circularized probe set. In some embodiments, the biological sample is contacted with the oligonucleotide and with the RNase H simultaneously or sequentially (in either order) before contacting the sample with the probe set. In any of the embodiments herein, the biological sample can be contacted with the RNase H (and optionally with the nucleic acid oligonucleotide) after formation of the circularized probe set. In any of the embodiments herein, the RNase H comprises an RNase H1 and/or an RNase H2. In some embodiments, RNase inactivating agents or inhibitors are added to the sample after cleaving the target RNA.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801).

In some embodiments, the RCP is generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some embodiments, the polymerase is phi29 DNA polymerase.

In some aspects, during the amplification step, modified nucleotides are added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) are anchored to a polymer matrix. For example, the polymer matrix can be a hydrogel. In some embodiments, one or more of the polynucleotide probe(s) is modified to contain functional groups that can be used as an anchoring site to attach the polynucleotide probes and/or amplification product to a polymer matrix. Exemplary modification and polymer matrix that can be employed in accordance with the provided embodiments comprise those described in, for example, U.S. Pat. No. 10,138,509B2, U.S. Pat. No. 10,266,888B2, US 2016/0024555, US 2018/0251833 and US 2017/0219465, each of which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

In some embodiments, the amplification products (e.g., RCPs) are immobilized within the matrix generally at the location of the nucleic acid being amplified, thereby creating a localized colony of amplicons. In some embodiments, the amplification products are immobilized within the matrix by steric factors. In some embodiments, the amplification products are immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products (e.g., RCPs) are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. In some embodiments, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination.

The methods disclosed herein allow for compaction of amplification products, wherein the self-complementary sequence is able to form hairpin structures, thereby reducing the overall size of the amplification products. Compaction of amplification products such as a rolling circle amplification product (RCP) without the introduction of exogenously added oligonucleotide probes in situ may provide a number of advantages. For instance, exogenously added oligonucleotide compaction probes may be stripped away along with detection probes following rounds of in situ hybridization and target detection. Therefore, strategies for persistent compaction of RCPs that are resistant to stripping are needed. In some aspects, compaction of RCPs by forming hairpin structures within the RCP results in compacted RCPs that enhance RCP detection, as reduction in size results in local concentration of the detection probes to increase signal intensity. In some embodiments, compaction also creates RCPs with increase stability and increased resolution of RCPs into discrete puncta, e.g., in a biological sample in situ.

### C. Detection and Analysis

In some aspects, the provided methods involve analyzing, e.g., detecting or determining, one or more sequences present in the probe sets or products thereof (e.g., rolling circle amplification products thereof). In some embodiments, the detecting is performed at one or more locations in the biological sample. In some embodiments, the locations are the locations of target nucleic acids (such as RNA transcripts) in the biological sample. In some embodiments, the locations are the locations at which the probe sets hybridize to the target nucleic acids (such as RNA transcripts) in the biological sample, and are optionally ligated and amplified by rolling circle amplification. In some instances, a sequence of the RCP is detected in the biological sample.

In some embodiments, detecting the one or more sequences present in the probe sets or complements thereof in the biological sample is performed, and the detected sequences are compared to an expected set of detected sequences. In some embodiments, the expected set of sequences is based on the barcode sequences of probe sets (e.g., barcode sequences of the first polynucleotide and/or the second polynucleotide) and the known expression levels of the RNA transcripts of the first, second, and/or third sets of genes in the first and second cell populations. In some embodiments, the one or more sequences are one or more barcode sequences or complements thereof. In some embodiments, the expected set of detected sequences include sequences expected to be detected at a high expression level (e.g., more than 20 counts of the detected sequence per cell) in the first and second cell populations. In some embodiments, the expected set of detected sequences include sequences expected to be detected at a medium expression level (e.g., 5-20 counts of the detected sequence per cell) in one or both of the first and second cell populations. In some embodiments, the expected set of detected sequences include sequences expected to be detected at a low expression level (e.g., 1-5 counts of the detected sequence per cell) in one or both of the first and second cell populations.

In some embodiments, the detecting comprises a plurality of repeated cycles of hybridization and removal of probes (e.g., detectably labeled probes, or intermediate probes that bind to detectably labeled probes) to the primary probe set (e.g., the first polynucleotide and the second polynucleotide) hybridized to the target nucleic acid, or to a rolling circle amplification product generated from the probe set hybridized to the target nucleic acid.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization assays, or sequencing by hybridization).

In some embodiments, the detecting can comprise binding an intermediate probe directly or indirectly to the primary probe set (e.g., the first polynucleotide and the second polynucleotide), binding a detectably labeled probe directly or indirectly to a detection region of the intermediate probe, and detecting a signal associated with the detectably labeled probe. In some embodiments, the method comprises detecting a rolling circle amplification product (RCP) generated using a circularized probe (such as those described in Section II.A). In some embodiments, the method comprises detecting a rolling circle amplification product (RCP) generated using a circularized probe. In some embodiments, detecting the RCP comprises binding an intermediate probe directly or indirectly to the RCP, binding a detectably labeled probe directly or indirectly to a detection region of the intermediate probe, and detecting a signal associated with the detectably labeled probe. In some embodiments, the method can comprise performing one or more wash steps to remove unbound and/or nonspecifically bound intermediate probe molecules from the circularized probes or the products of the circularized probes.

In some embodiments, the detecting comprises detecting signals associated with detectably labeled probes that are hybridized to barcode regions or complements thereof in the probe set (e.g., the first polynucleotide and the second polynucleotide as described in Section II.A.) or a product thereof (e.g., an RCP); and/or detecting signals associated with detectably labeled probes that are hybridized to intermediate probes which are in turn hybridized to the barcode regions or complements thereof. In some embodiments, the detectably labeled probes is fluorescently labeled.

In some embodiments, the methods comprise detecting the sequence in all or a portion of a probe set (e.g., the first polynucleotide and the second polynucleotide) or an RCP, or detecting a sequence of the probe set or RCP, such as one or more barcode sequences present in the probe set or RCP. In some embodiments, the sequence of the RCP, or barcode thereof, is indicative of a sequence of the target nucleic acid to which the RCP is hybridized. In some embodiments, the analysis and/or sequence determination comprises detecting a sequence in all or a portion of the nucleic acid concatemer and/or in situ hybridization to the RCP. In some embodiments, the detection step involves sequencing by hybridization, sequencing by ligation, sequencing by synthesis, sequencing by binding, and/or fluorescent in situ sequencing (FISSEQ), and/or hybridization-based in situ sequencing. In some embodiments, the detection step is by sequential fluorescent in situ hybridization (e.g., for combinatorial decoding of the barcode sequence or complement thereof).

In some embodiments, the detection or determination comprises hybridizing to the a probe directly or indirectly a detection oligonucleotide labeled with a fluorophore, an isotope, a mass tag, or a combination thereof. In some embodiments, the detection or determination comprises imaging the probe associated with the target nucleic acid (e.g., imaging one or more detectably labeled probes hybridized thereto). In some embodiments, the target nucleic acid is an mRNA in a tissue sample, and the detection or determination is performed when the target nucleic acid and/or the amplification product is in situ in the tissue sample. In some embodiments, the target nucleic acid is associated with an amplification product (e.g., a rolling circle amplification product) that is detected.

In some aspects, the provided methods comprise imaging a detectably labeled probe bound directly or indirectly to the probe set or product thereof and detecting the detectable label. In some embodiments, the detectably labeled probe comprises a detectable label that can be measured and quantitated. The label or detectable label can comprise a directly or indirectly detectable moiety, e.g., any fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in biological samples (e.g., tissue samples) can often be hindered by the presence of strong background fluorescence. Background fluorescence can include autofluorescence (that can arise from a variety of sources, including aldehyde fixation, extracellular matrix components, red blood cells, lipofuscin, and the like), as opposed to the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, acquorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259(1991). In some embodiments, exemplary techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labeling can also be carried out with quantum dots, as described in U.S. Pat. No. 6,322,901, U.S. Pat. No. 6,576,291, U.S. Pat. No. 6,423,551, U.S. Pat. No. 6,251,303, U.S. Pat. No. 6,319,426, U.S. Pat. No. 6,426,513, U.S. Pat. No. 6,444,143, U.S. Pat. No. 5,990,479, U.S. Pat. No. 6,207,392, US 2002/0045045 and US 2003/0017264, all of which are herein incorporated by reference in their entireties. In some embodiments, a fluorescent label comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Examples of methods for custom synthesis of nucleotides having other fluorophores are described in Henegariu et al. (2000) Nature Biotechnol. 18:345.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610,647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycocrythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection.

Other suitable labels for a polynucleotide sequence may comprise fluorescein (FAM), digoxigenin, dinitrophenol (DNP), dansyl, biotin, bromodeoxyuridine (BrdU), hexahistidine (6xHis) (SEQ ID NO: 2), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a oligonucleotide sequence is indirectly labeled, especially with a hapten that is then bound by a capture agent, e.g., as disclosed in U.S. Pat. Nos. 5,344,757, 5,702,888, 5,354,657, 5,198,537 and 4,849,336, and 5,073,562, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity-so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, the assay comprises in situ sequencing. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932, all of which are herein incorporated by reference in their entireties. Exemplary techniques for in situ sequencing or in in situ sequence detection comprise, but are not limited to, STARmap (described for example in Wang et al., (2018) Science, 361(6499) 5691, the content of which is herein incorporated by reference in its entirety), MERFISH (described for example in Moffitt, (2016) Methods in Enzymology, 572, 1-49, the content of which is herein incorporated by reference in its entirety), hybridization-based in situ sequencing (HybISS) (described for example in Gyllborg et al., Nucleic Acids Res (2020) 48(19):e112, the content of which is herein incorporated by reference in its entirety), and FISSEQ (described for example in US 2019/0032121, the content of which is herein incorporated by reference in its entirety).

In some embodiments, analyzing, e.g., detecting or determining, one or more sequences present in the biological sample is performed using a base-by-base sequencing method, e.g., sequencing-by-synthesis (SBS), sequencing-by-avidity (SBA) or sequencing-by-binding (SBB). In some embodiments, the biological sample is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

Generally in sequencing-by-synthesis methods, a first population of detectably labeled nucleotides (e.g., dNTPs) are introduced to contact a template nucleotide (e.g., a barcode sequence in the RCP) hybridized to a sequencing primer, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced, but in order for a second detectably labeled nucleotide to incorporate into the extended sequencing primer, nucleotides in the first population of nucleotides that have not incorporated into a sequencing primer are generally removed (e.g., by washing), and a second population of detectably labeled nucleotides are introduced into the reaction. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in some embodiments, cycles of introducing and removing detectably labeled nucleotides are performed.

In some embodiments, the base-by-base sequencing comprises using a polymerase that is fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

In some embodiments, sequencing is performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Example SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing is performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, sequencing is performed by sequencing-by-avidity (SBA). Some aspects of SBA approaches are described in U.S. Pat. No. 10,768,173 B2, the contents of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

In some embodiments, detection of the barcode sequences is performed by sequential hybridization of probes to the barcode sequences or complements thereof and detecting complexes formed by the probes and barcode sequences or complements thereof. In some cases, each barcode sequence or complement thereof is assigned a sequence of signal codes that identifies the barcode sequence or complement thereof (e.g., a temporal signal signature or code that identifies the analyte), and detecting the barcode sequences or complements thereof can comprise decoding the barcode sequences of complements thereof by detecting the corresponding sequences of signal codes detected from sequential hybridization, detection, and removal of sequential pools of intermediate probes and the universal pool of detectably labeled probes. In some cases, the sequences of signal codes comprise fluorophore sequences assigned to the corresponding barcode sequences or complements thereof. In some embodiments, the detectably labeled probes are fluorescently labeled. In some embodiments, the barcode sequence or complement thereof is performed by sequential probe hybridization as described in US 2021/0340618, the content of which is herein incorporated by reference in its entirety.

In any of the embodiments herein, the detecting step can comprise contacting the biological sample with one or more detectably labeled probes that directly or indirectly hybridize to the barcode sequences or complements thereof (e.g., in amplification products generated using the probe sets), and dehybridizing the one or more detectably labeled probes. In any of the embodiments herein, the contacting and dehybridizing steps is repeated with the one or more detectably labeled probes and/or one or more other detectably labeled probes that directly or indirectly hybridize to the barcode sequences or complements thereof. In some aspects, the method comprises sequential hybridization of detectably labeled probes to create a spatiotemporal signal signature or code that identifies the analyte.

In any of the embodiments herein, the detecting step can comprise contacting the biological sample with one or more first detectably labeled probes that directly hybridize to the plurality of probes or probe sets. In some instances, the detecting step can comprise contacting the biological sample with one or more first detectably labeled probes that indirectly hybridize to the plurality of probes or probe sets. In any of the embodiments herein, the detecting step can comprise contacting the biological sample with one or more first detectably labeled probes that directly or indirectly hybridize to the plurality of probes or probe sets.

In any of the embodiments herein, the detecting step comprises contacting the biological sample with one or more intermediate probes that directly or indirectly hybridize to the barcode sequences or complements thereof (e.g., of the first polynucleotide and/or the second polynucleotide or rolling circle amplification product generated using the first polynucleotide and the second polynucleotide), wherein the one or more intermediate probes are detectable using one or more detectably labeled probes. In any of the embodiments herein, the detecting step can further comprise dehybridizing the one or more intermediate probes and/or the one or more detectably labeled probes from the barcode sequences or complements thereof (e.g., of the first polynucleotide and/or the second polynucleotide or rolling circle amplification product generated using the first polynucleotide and the second polynucleotide). In any of the embodiments herein, the contacting and dehybridizing steps can be repeated with the one or more intermediate probes, the one or more detectably labeled probes, one or more other intermediate probes, and/or one or more other detectably labeled probes. In some cases, the repeated contacting, detection and dehybridizing steps allows detection of barcode sequences or complements thereof and identification of the corresponding sequences of signal codes (e.g., fluorophore sequences assigned to the corresponding barcode sequences or complements thereof).

In some embodiments, sequencing is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309:1728-1732, and in U.S. Pat. No. 5,599,675; 5,750,341; 6,969,488; 6,172,218; and 6,306,597, all of which are herein incorporated by reference in their entireties.

In some embodiments, nucleic acid hybridization is used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877(2004), all of which are herein incorporated by reference in their entireties.

In some embodiments, sequential rounds of nucleic acid hybridization is used for sequencing is employed for detection. In each round of nucleic acid hybridization, labeled nucleic acid decoder probes that are complementary to a portion of a barcode sequence (e.g., a sub-barcode) are utilized. In some embodiments, the sub-barcodes are overlapping.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some embodiments, the analysis and/or sequence determination involves washing to remove unbound polynucleotides, thereafter revealing a fluorescent product for imaging.

### III. Samples, Analytes, and Target Sequences

### A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaca, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

In some embodiments, the biological sample includes any number of macromolecules, for example, cellular macromolecules and organelles (e.g., mitochondria and nuclei). In some embodiments, the biological sample comprises nucleic acids (such as DNA or RNA), proteins/polypeptides, carbohydrates, and/or lipids. In some embodiments, the biological sample is obtained as a tissue sample, such as a tissue section, biopsy, a core biopsy, needle aspirate, or fine needle aspirate. In some embodiments, the biological sample is or comprise a cell pellet or a section of a cell pellet. In some embodiments, the biological sample is or comprise a cell block or a section of a cell block. In some embodiments, the sample comprises a fluid sample, such as a blood sample, urine sample, or saliva sample. In some embodiments, the sample comprises a skin sample, a colon sample, a check swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample comprises cells which are deposited on a surface.

In some embodiments, biological samples are derived from a homogeneous culture or population of the subjects or organisms mentioned herein or alternatively from a collection of several different organisms. In some embodiments, biological samples include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, a substrate herein is any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section is prepared by applying a touch imprint of a biological sample to a suitable substrate material.

In some embodiments, the thickness of the tissue section is a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analyzed.

In some embodiments, multiple sections are obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analyzed successively to obtain three-dimensional information about the biological sample.

In some embodiments, the biological sample (e.g., a tissue section as described above) is prepared by deep freezing at a temperature suitable to maintain or preserve the integrity (e.g., the physical characteristics) of the tissue structure. In some embodiments, the frozen tissue sample is sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample is prepared using formalin-fixation and paraffin-embedding (FFPE). In some embodiments, cell suspensions and other non-tissue samples are prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes). In some embodiments, the biological sample (e.g., FFPE sample) is permeable after deparaffinization. In some embodiments, processing of the biological sample, such as de-waxing, allows the biological sample to become permeabilized.

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circularizable probe set (e.g., the first polynucleotide and the second polynucleotide). In some embodiments, one or more post-fixing step is performed after a hybridization complex comprising a probe and a target is formed in a sample. In some embodiments, one or more post-fixing step is performed prior to a ligation reaction disclosed herein.

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

In some embodiments, a biological sample is permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference in its entirety. Any suitable method for sample permeabilization can generally be used in connection with the samples described herein.

In some embodiments, the biological sample is permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, is added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ii) Embedding

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample is embedded by contacting the sample with a suitable polymer material, and activating the polymer material to form a hydrogel. In some embodiments, the hydrogel is formed such that the hydrogel is internalized within the biological sample. Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) are embedded in a 3D matrix. In some embodiments, a 3D matrix comprises a network of natural molecules and/or synthetic molecules that are chemically and/or enzymatically linked, e.g., by crosslinking. In some embodiments, a 3D matrix comprises a synthetic polymer. In some embodiments, a 3D matrix comprises a hydrogel.

In some aspects, a biological sample is embedded in any of a variety of other embedding materials to provide structural substrate to the sample prior to sectioning and other handling steps. In some cases, the embedding material is removed e.g., prior to analysis of tissue sections obtained from the sample. Suitable embedding materials include, but are not limited to, waxes, resins (e.g., methacrylate resins), epoxies, and agar.

In some embodiments, the biological sample is embedded in a matrix (e.g., a hydrogel matrix). Embedding the sample in this manner typically involves contacting the biological sample with a hydrogel such that the biological sample becomes surrounded by the hydrogel. In some embodiments, the sample is embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

In some embodiments, the biological sample is reversibly cross-linked prior to or during an in situ assay. In some aspects, the analytes, polynucleotides and/or amplification product (e.g., amplicon) of an analyte or a probe bound thereto are anchored to a polymer matrix In some embodiments, the polymer matrix comprises a hydrogel. In some embodiments, one or more of the polynucleotide probe(s) and/or amplification product (e.g., amplicon) thereof are modified to contain functional groups that can be used as an anchoring site to attach the polynucleotide probes and/or amplification product to a polymer matrix. In some embodiments, a modified probe comprising oligo dT is used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel comprises hydrogel subunits, such as, but not limited to, acrylamide, bis-acrylamide, polyacrylamide and derivatives thereof, poly (ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly (hydroxyethyl acrylate), and poly (hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference in their entireties.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the content of which is herein incorporated by reference in its entirety.

In some embodiments, the hydrogel forms the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible. In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some embodiments, optical labels are added to the hydrogel subunits before, contemporancously with, and/or after polymerization.

In some embodiments, hydrogels embedded within biological samples are cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a biological sample embedded in a matrix (e.g., a hydrogel) is isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9×its non-expanded size. In some embodiments, the sample is isometrically expanded to at least 2× and less than 20× of its non-expanded size.

(iii) Staining and Immunohistochemistry (IHC)

To facilitate visualization, in some embodiments, biological samples are stained using a wide variety of stains and staining techniques. In some embodiments, for example, a sample is stained using any number of stains and/or immunohistochemical reagents. In some embodiments, one or more staining steps are performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain is specific to proteins, phospholipids, DNA (e.g., dsDNA, ssDNA), RNA, an organelle or compartment of the cell. In some embodiments, the sample is contacted with one or more labeled antibodies (e.g., a primary antibody specific for the analyte of interest and a labeled secondary antibody specific for the primary antibody). In some embodiments, cells in the sample is segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, cosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample is stained with haematoxylin and cosin (H&E).

In some embodiments, the sample are stained using hematoxylin and cosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples is destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference in their entireties.

### B. Analytes

A biological sample may comprise one or a plurality of analytes of interest. Methods for performing multiplexed assays to analyze two or more different analytes in a single biological sample are provided. The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte is directly or indirectly detected.

In some embodiments, analytes are derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaccous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. In some embodiments, the analyte is a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo-or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte is an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. In some embodiments, the RNA comprises circular RNA. In some embodiments, the RNA is a bacterial rRNA (e.g., 16s rRNA or 23s rRNA).

In some embodiments described herein, an analyte is a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample.

### (ii) Labeling Agents

In some embodiments, provided herein are methods and compositions for analyzing endogenous analytes (e.g., RNA, ssDNA, cell surface or intracellular proteins, and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes a barcode that is associated with or otherwise identifies the analyte binding moiety. In some embodiments, by identifying an analyte binding moicty by identifying its associated analyte binding moicty barcode, the analyte to which the analyte binding moiety binds can also be identified. An analyte binding moiety barcode can be a nucleic acid sequence of a given length and/or sequence that is associated with the analyte binding moicty. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moicty capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, sec, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moicty includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moicties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, the content of which is herein incorporated by reference in its entirety. Likewise, protein and peptide biotinylation techniques have been developed and are readily available. See, e.g., U.S. Pat. No. 6,265,552, the content of which is herein incorporated by reference in its entirety. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. In some embodiments, the label is conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moicty (ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moicty. Results of protein analysis in a sample (e.g., a tissue sample or a cell) can be associated with DNA and/or RNA analysis in the sample.

### C. Barcode Sequences

In some embodiments, an analyte described herein is associated with one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In any of the preceding embodiments, the methods provided herein include analyzing (e.g., detecting or determining) the barcodes by sequential hybridization and detection with a plurality of labelled probes (e.g., detection oligos).

In some embodiments, in a barcode detection or sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, sec, e.g., U.S. Pat. Pub. 20190055594 and U.S. Pat. Pub 20210164039, which are hereby incorporated by reference in their entirety.

### IV. Compositions, Systems and Kits

In some aspects, provided herein are compositions comprising any of the probe sets comprising a first polynucleotide and a second polynucleotide described herein. Also provided herein are kits, for analyzing (e.g., detecting or determining) an analyte in a biological sample according to any of the methods described herein. In some embodiments, provided herein is a kit comprising any of the probe sets comprising a first polynucleotide and a second polynucleotide described herein. The various components of the kit may be present in separate containers or certain compatible components may be pre-combined into a single container. In some embodiments, the kits further contain instructions for using the components of the kit to practice the provided methods.

Provided herein is a system comprising any of the probe sets comprising a first polynucleotide and a second polynucleotide described herein (e.g., in Section II). In some instances, the system comprise a probe set comprising a first polynucleotide and a second polynucleotide, wherein the first polynucleotide comprises a first target binding region and a stem region, wherein the stem region is at one terminus of the first polynucleotide and comprises a second polynucleotide binding sequence and a self-complementary sequence, the second polynucleotide comprises a second target binding region and a second probe region, wherein the second probe region is configured to bind to the second polynucleotide binding sequence of the first polynucleotide; a ligase for generating a circularized probe comprising the first polynucleotide and the second polynucleotide; and a polymerase for performing rolling circle amplification (RCA) of the circularized probe. In some embodiments, the system comprises reagents for detecting a sequence of the first polynucleotide and the second polynucleotide or a complement thereof (e.g., as described in Section II). In some instances, the reagents for detecting the sequence of the first polynucleotide and the second polynucleotide or a complement thereof comprises one or more detectably-labeled probes. In some instances, the reagents for detecting the sequence of the first polynucleotide and the second polynucleotide or a complement thereof comprises reagents for performing sequencing-by-synthesis (SBS), sequencing-by-avidity (SBA) or sequencing-by-binding (SBB).

In some embodiments, the systems or kits comprise reagents and/or consumables required for performing one or more steps of the provided methods. In some embodiments, the systems or kits contain reagents for fixing, embedding, and/or permeabilizing the biological sample. In some embodiments, the systems or kits contain reagents, such as enzymes and buffers for ligation and/or amplification, such as ligases and/or polymerases. In some aspects, the system or kit can also comprise any of the reagents described herein, e.g., wash buffer and ligation buffer. In some embodiments, the systems or kits contain reagents for detection and/or sequencing, such as barcode detection probes or detectable labels. In some embodiments, the kits optionally contain other components, for example nucleic acid primers, ligases, and polymerases.

### V. Opto-Fluidic Instruments for Analysis of Biological Samples

Provided herein is an instrument having integrated optics and fluidics modules (an “opto-fluidic instrument” or “opto-fluidic system”) for detecting target molecules (e.g., nucleic acids, proteins, antibodies, etc.) in biological samples (e.g., one or more cells or a tissue sample) as described herein. In an opto-fluidic instrument, the fluidics module is configured to deliver one or more reagents (e.g., detectably labeled probes) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more probing cycles (e.g., as described in Section II). In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules in their naturally occurring place (i.e., in situ) within the biological sample. For example, an opto-fluidic instrument may be an in-situ analysis system used to analyze a biological sample and detect target molecules including but not limited to DNA, RNA, proteins, antibodies, and/or the like.

It is to be noted that, although the above discussion relates to an opto-fluidic instrument that can be used for in situ target molecule detection via probe hybridization, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

### VI. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some instances, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Probe Design for Splint-Free Ligation

This Example describes the design of a probe set comprising a first polynucleotide and a second polynucleotide for splint-free ligation to form a circularized probe for in situ analysis of target nucleic acid sequences, such as mRNA sequences. The probe set permits highly efficient ligation, as the probe set does not require the use of a splint, which can prevent the ligation when both ends to be ligated are occupied by splints. The probe set also permits generation of compacted RCPs by using self-complementary sequences within the RCP that are able to form hairpin structures, therefore increasing the detection resolution.

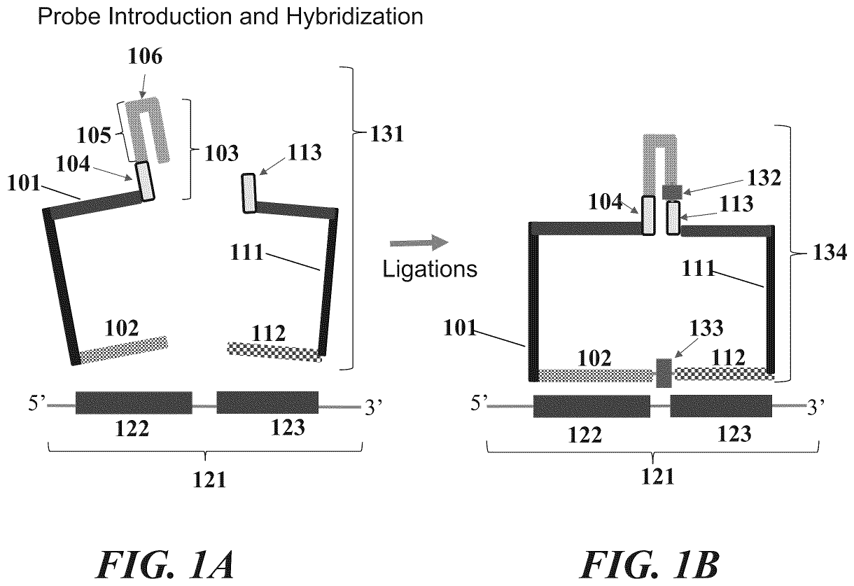

As shown in FIG. 1A, the first polynucleotide 101 in the probe set 131 comprises a first target binding region 102 and a stem region 103. The stem region 103 is at one terminus of the first polynucleotide 101 (e.g., at the 3′ end of the first polynucleotide), and comprises a self-complementary sequence 105, which can comprise a first portion and a second portion that are complementary to each other and a linker 106 linking the first portion and the second portion. The stem region 103 also comprises a second polynucleotide binding sequence 104. As shown in FIG. 1A, the second polynucleotide 111 in the probe set 131 comprises a second target binding region 112 and a second probe region 113.

The first polynucleotide and the second polynucleotide bind to a target nucleic acid sequence 121, such as an mRNA, at a location in the biological sample. Specifically, the first target binding region 102 binds to a first target region 122 in a target nucleic acid 121 at a location in the biological sample and the second target binding region 112 binds to a second target region 123 in the target nucleic acid 121. Upon hybridization, the ends of the first target binding region 102 and the second target binding region 112 (such as the 5′ end of the first target binding region and the 3′ end of the second target binding region) are adjacent to each other, and connected by ligation 133 using the target nucleic acid as a template.

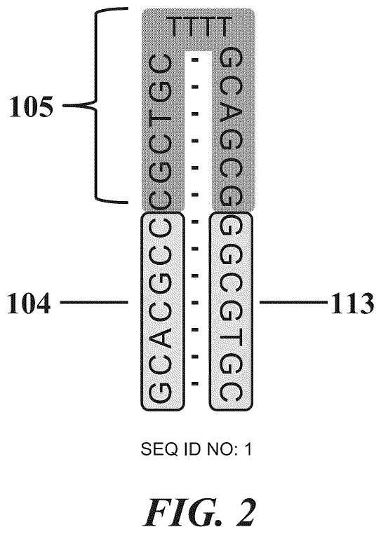

Additionally, the second probe region 113 and the second polynucleotide binding sequence 104 hybridize to each other (FIG. 2). The complementary sequence 105 forms a hairpin structure by self hybridization (FIG. 2; SEQ ID NO:1). As a result, a terminus (such as a 3′ end) of the first polynucleotide is brought adjacent to a terminus (such as a 5′ end) of the second polynucleotide. The termini are then connected by ligation 132 using the first polynucleotide as a template. In some cases, the orientation of the first polynucleotide 101 in the probe set 131 is reversed with the 5′ end binding the target nucleic acid sequence. In some cases, instead of the first polynucleotide, the second polynucleotide comprises the second probe region 113 and a self-complementary sequence comprising a first portion and a second portion that are complementary to each other to form a hairpin.

### Example 2: Hybridization, Amplification and Detection

Probe sets as described in Example 1 above are hybridized to form a circularized probe, amplified by rolling circle amplification to form a rolling circle product (RCP), and detected in a biological sample (such as a tissue section) for in situ analysis of target nucleic acids.

A library of different probe sets targeting different target nucleic acids, or different probe sets targeting the same nucleic acid at different positions, are pooled. The probe mixture is heated, then cooled down to room temperature, and incubated with a cell culture sample or a thin tissue section sample and hybridization buffer, for hybridization to target nucleic acids in the sample.

The sample is washed, then incubated with a T4 DNA ligase for a first ligation (132 of FIG. 1A) and a second ligation (133 of FIG. 1A) at room temperature, after which a circularized probe is formed (FIG. 1B). In some cases, the first and second ligations are performed at the same time. In some cases, the first ligation is performed, the sample is washed, then the second ligation is performed. The sample is washed and then incubated with a rolling-circle amplification (RCA) mixture containing a Phi29 DNA polymerase and dNTPs, and incubated at approximately 30° C. for RCA of the circularized probe. In some instances, modified nucleotide bases (e.g., 5-(3-aminoallyl)-dUTP) is included in the RCA mixture. After hybridization, ligation and amplification, the probe set, RCA amplification product (also called “amplicon”) and in some instances the target nucleic acid, can form a complex or a structure, such as a nanometer scale nucleic acid ball (also called “nanoball”). Multiple hairpin structures form in the RCP with copies of the self-complementary sequence, reducing the RCP size.

For single-gene detection, fluorescently labeled oligonucleotides complementary to a portion of the RCP are incubated with the sample, washed and images are obtained.

For sequencing, the sample is treated with stripping buffer, washed, and incubated with a sequencing mixture containing a T4 DNA ligase, fluorescently labeled sequencing oligonucleotides, and images are obtained. Multiple cycles of sequencing is performed, and DAPI and Nissl staining can also be performed. Images are acquired using a confocal microscope.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for analyzing a biological sample, comprising:
(a) contacting the biological sample with a probe set comprising a first polynucleotide and a second polynucleotide, wherein:
the first polynucleotide comprises a first target binding region and a stem region, wherein the stem region is at one terminus of the first polynucleotide and comprises a second polynucleotide binding sequence and a self-complementary sequence,
the second polynucleotide comprises a second target binding region and a second probe region, wherein the second probe region binds to the second polynucleotide binding sequence of the first polynucleotide,
the stem region brings a terminus of the first polynucleotide adjacent to a terminus of the second polynucleotide, and
the first target binding region binds to a first target region in a target nucleic acid at a location in the biological sample and the second target binding region binds to a second target region in the target nucleic acid;
(b) connecting the first polynucleotide and the second polynucleotide to generate a circularized probe at the location in the biological sample, wherein the connecting comprises:
(i) ligating the first target binding region and the second target binding region using the target nucleic acid as a template, and
(ii) ligating the stem region and the second probe region using the first polynucleotide as a template;
(c) using a polymerase to perform rolling circle amplification (RCA) of the circularized probe, thereby generating a rolling circle amplification product (RCP), wherein the polymerase is optionally a Phi29 polymerase; and
(d) detecting the RCP at the location in the biological sample, thereby detecting the target nucleic acid or a sequence thereof in the biological sample.

2-9. (canceled)

10. The method of claim 1, wherein the self-complementary sequence further comprises a linker.

11-12. (canceled)

13. The method of claim 10, wherein the linker comprises a homopolymeric sequence, optionally wherein the linker comprises a poly (dt) sequence.

14-28. (canceled)

29. The method of claim 1, wherein the first polynucleotide and/or the second polynucleotide comprises a barcode region.

30. The method of claim 29, wherein the barcode region in the first polynucleotide is nonoverlapping with the stem region, and/or the barcode region in the second polynucleotide is nonoverlapping with the second probe region.

31. The method of claim 29, wherein the barcode region in the first polynucleotide is at least partially overlapping with the first probe region, and/or the barcode region in the second polynucleotide is at least partially overlapping with the second probe region.

32. The method of claim 1, wherein the biological sample is contacted with a plurality of probe sets each comprising a first polynucleotide and a second polynucleotide that target a different target nucleic acid.

33. The method of claim 32, wherein the stem regions in the plurality of probe sets are common among first polynucleotides targeting different target nucleic acids, and/or the second probe regions in the plurality of probe sets are common among second polynucleotides targeting different target nucleic acids.

34. The method of claim 32, wherein the stem regions in the plurality of probe sets are each associated with a different target nucleic acid, and/or the second probe regions in the plurality of probe sets are each associated with a different target nucleic acid.

35. The method of claim 1, wherein:
the first polynucleotide comprises, from 5′ to 3′: the first target binding region, an optional first spacer, the second polynucleotide binding sequence and the self-complementary sequence; and
the second polynucleotide comprises, from 3′ to 5′: the second target binding region, an optional second spacer, and the second probe region.

36-41. (canceled)

42. The method of claim 1, wherein the ligation in (b)(i) is an RNA-templated ligation and the ligation in (b)(ii) is a DNA-templated ligation.

43-46. (canceled)

47. The method of claim 1, further comprising contacting the biological sample with a primer prior to the rolling circle amplification in (c).

48. The method of claim 1, wherein the target nucleic acid acts as a primer for the rolling circle amplification in (c).

49. The method of claim 1, wherein the method further comprises imaging the biological sample to detect the circularized probe and/or the RCP thereof.

50. The method of claim 49, wherein the imaging comprises detecting a signal associated with the circularized probe and/or the RCP thereof at the location in the biological sample.

51. The method of claim 29, wherein the circularized probe and/or the RCP thereof comprise the one or more barcode sequences or complements thereof.

52. The method of claim 51, wherein the one or more barcode sequences or complements thereof are detected by: contacting the biological sample with one or more detectably-labeled probes that directly or indirectly hybridize to the one or more barcode sequences or complements thereof, and detecting signals associated with the one or more detectably-labeled probes.

53. The method of claim 1, wherein the biological sample is a tissue sample.

54. The method of claim 53, wherein the target nucleic acid is in a cell in the tissue sample.

55-58. (canceled)

59. The method of claim 58, wherein the target nucleic acid is cDNA or mRNA.

60-73. (canceled)


SEQUENCE LISTING (2 sequences)

SEQ ID NO: 1 - DNA, 30 bp, synthetic construct
  source 1..30: mol_type = other DNA
gcacgcccgctgcttttgcagcgggcgtgc

SEQ ID NO: 2 - protein, 6 aa, synthetic construct
HHHHHH